(19) United States
(12) Patent Application Publication
(10) Pub. No.: US 2022/0254450 A1
(43) Pub. Date: Aug. 11, 2022

(54) method for classifying individuals in mixtures of DNA and its deep learning model

(71) Applicant: National Taiwan University, Taipei (TW)

(72) Inventors: Mong-Hsun Tsai, Taipei (TW); Eric Y Chuang, Taipei (TW); Hsiao-Lin Hwa, Taipei (TW); Nam Nhut Phan, Taipei (TW)

(21) Appl. No.: 17/550,380

(22) Filed: Dec. 14, 2021

Related U.S. Application Data
(60) Provisional application No. 63/147,520, filed on Feb. 9, 2021.

(51) Int. Cl. G16B 40/00; G16B 30/00; G06N 3/08

(52) U.S. Cl. CPC G16B 40/00; G06N 3/08; G16B 30/00

(57) ABSTRACT

A method for classifying individuals in mixtures of DNA is disclosed. The method comprises: Provide next-generation sequencing (NGS) data which comprises raw sequence reads originated from mixtures of DNA; performing a data processing procedure to generate a plurality of sparse matrix; and input the plurality of sparse matrix into a trained deep learning model installed on computers to classify individuals in the mixtures of DNA. In particular, the method is used to classify individuals in mixture of the DNAs from forensic dataset or whole exome sequencing dataset.

### TECHNICAL FIELD OF THE INVENTION

The invention relates to a method for classifying individuals in mixtures of DNA. In particular, the method describes a sliding window trimming procedure and high performance deep learning model to classify individuals in mixtures of DNA through using next generation sequencing data.

### BACKGROUND OF THE INVENTION

The last decade has witnessed acceleration in the development and use of high-throughput data analysis techniques in biomedical research. Large-scale access to digital data and computing infrastructure has led to artificial intelligence (AI) being widely applied to various research fields. Application of such methods in biomedical research, including pathological image analysis, radiomic data analysis, and genomic data analysis, is on the rise.

In the past few years, numerous studies have been conducted on next-generation sequencing (NGS) data to conduct classification or infer genotypes or phenotypes. However, in forensic studies, identifying individuals from a mixture of more than two genetic contributors can be a significant challenge. The difficulty is further enhanced for mixtures with highly imbalanced major and minor contributors. In addition to forensic studies, there has been an explosion of studies in cancer research, owing to the big data from this field, and several models have been developed for different research purposes. A majority of these studies used biomedical imaging data such as histological images and magnetic resonance images. However, these models either require pre-selection of useful variants prior to training, displaying higher misclassification rate for organs that share a common developmental origin, or are limited to binary classification tasks. They also use receiver operating characteristic curves (ROCs) as the metric for performance evaluation, which is not precise enough to thoroughly evaluate model performance, and is unable to achieve sufficient accuracy to be completely confident about the classification. In another study, features extracted from multi-omics data were used to train a model; however, this approach required domain knowledge and feature curation before feeding to the model. Such features may vary between patients, so this approach is limited by feature bias.

Therefore, none of the existing methods are suitable for classification of the types of genomic data currently being generated by the medical and forensic fields.

### SUMMARY OF THE INVENTION

Based on the aforementioned requirement of classification of the types of genomic data in biomedical and forensic industry, the invention discloses a method for classifying individuals in mixtures of DNA by using a deep learning (DL) model. The deep learning model can automatically and globally select features using convolutional layers from raw next-generation sequencing (NGS) data could provide a robust solution for non-biased feature selection. Briefly, the goal of this invention is to develop a method and DL pipeline for NGS data classification that can achieve accuracy as high as 97% and can operate across multiple disciplines.

In one aspect, the present invention discloses a 1-dimensional deep convolutional neural network (CNN) that can potentially be applied globally to NGS data for the classification of individuals in mixtures of DNA. The invention provides a novel method and pipeline, including both sequencing data processing steps and ML steps. In the method, a sliding window method is applied to overcome the problem of raw NGS reads length variation, which leads to a dramatic improvement of the DL model performance.

In another aspect, to demonstrate the versatility of the invention, the invented method is able to identify individuals from highly imbalanced mixture of samples on a forensic dataset. The invention is also used on whole exome sequencing (WES) data from breast cancer patients to classify post-mastectomy patients into triple negative (TNBC) or luminal A subtypes. Accordingly, the invention provides a robust and highly accurate DL pipeline that is applicable across different NGS platforms.

In another aspect, the sequencing data processing comprises following steps for generating a plurality of sparse matrix: sliding window trimming, sorting and indexing, mapping, reverse complementation, and encoding to generate the plurality of sparse matrix. Preferably, multiple sequence length combinations or combinations based on at least 4 sparse matrices generated from the combined forward and reverse sequence reads with same length or different length are as inputs into the trained DL model for classifying individuals in the mixtures of DNA in the invention.

In another aspect, the DL model architecture is another facet that also plays an important role in achieving good model performance. In the invention, we used a 1-dimensional CNN which transformed sequence reads into higher abstraction levels using 2 blocks of convolutional layers. We use 2 fully connected layers with 1024 neurons and modified the number of convolutional layers, it proves that they had a higher impact on model performance.

In another aspect, the method further comprises a step for checking quality of the raw sequence reads, and phred33 score is used for measure of the quality of the raw sequence reads, and the raw sequence reads are trimmed if the phred33 score is below 15.

In another aspect, the trained deep learning model is a one-dimensional deep convolutional neural network constructed from a first convolution layer, a first batch normalization layer, a second convolution layer, a second batch normalization layer, a first max pooling layer, a first concatenate layer, a second max pooling layer, a first flatten layer, a second concatenate layer, a third batch normalization layer, a first hidden layer, a fourth batch normalization layer and a second hidden layer, wherein the first convolution layer connects to the first batch normalization layer, the first batch normalization layer connects to the second convolution layer, the second convolution layer connects to the second batch normalization layer, the second batch normalization layer connects to the first max pooling layer, the first max pooling layer connects to the first concatenate layer, the first concatenate layer connects to the second max pooling layer, the second max pooling layer connects to the first flatten layer, the first flatten layer connects to the second concatenate layer, the second concatenate layer connects to the third batch normalization layer, the third batch normalization layer connects to the first hidden layer, the first hidden layer connects to the fourth batch normalization layer, the fourth batch normalization layer connects to the second hidden layer, and wherein the second hidden layer outputs classification of individuals in the mixtures of DNA.

In another aspect, the method further comprises a step for validating the trained deep learning model, and the trained deep learning model has accuracy equal to or more than about 90%.

In another aspect, the method is to classify individuals in mixture of the DNAs from forensic dataset or whole exome sequencing dataset.

Accordingly, the invention is to provide a method of classifying individuals in mixtures of DNA. The method comprises: provide next-generation sequencing (NGS) data which comprises raw sequence reads originated from mixtures of DNA; perform a data processing procedure to generate a plurality of sparse matrix; and input the plurality of sparse matrix into a trained deep learning model installed on computers to classify individuals in the mixtures of DNA.

### BRIEF DESCRIPTION OF THE DRAWINGS

FIG. 1 is workflow of the method for classifying individuals in mixtures of DNA by using a deep learning (DL) model;

FIG. 2 is workflow of the sequencing data processing;

FIG. 3 is workflow for the DNA ratio prediction model, where the processing sequencing data from raw to final format is illustrated in FIG. 3(A); and overall schematic design of the multi-input 1-dimensional CNN is illustrated in FIG. 3B;

FIG. 4 is the sliding window trimming scheme of the invention;

FIG. 5 is the invented input strategy of the sparse matrices;

FIG. 6 is the confusion matrix plot of 10 different models trained with sequence reads trimmed by different sliding windows; and

FIG. 7 is precision-recall curve (A) and ROC curve (B) of the classification of TNBC and luminal A breast cancer subtype, respectively.

### BRIEF DESCRIPTION OF THE PREFERRED EMBODIMENTS

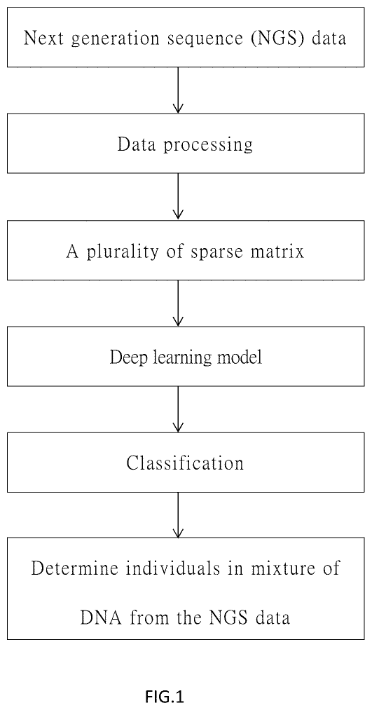

In one embodiment, the invention discloses a method of classifying individuals in mixtures of DNA and its workflow is shown in FIG. 1. The method comprises: provide next-generation sequencing (NGS) data which comprises raw sequence reads originated from mixtures of DNA; perform a data processing procedure to generate a plurality of sparse matrix; and input the plurality of sparse matrix into a trained deep learning model installed on computers to classify individuals in the mixtures of DNA.

In another embodiment, the data processing procedure is to sort and index the trimmed sequence reads prior to the mapping step.

In another embodiment, the method further comprises a step for checking quality of the raw sequence reads, and phred33 score is used for measure of the quality of the raw sequence reads, and the raw sequence reads are trimmed if the phred33 score is below 15.

In another embodiment, the trained deep learning model is a one-dimensional deep convolutional neural network constructed from a first convolution layer, a first batch normalization layer, a second convolution layer, a second batch normalization layer, a first max pooling layer, a first concatenate layer, a second max pooling layer, a first flatten layer, a second concatenate layer, a third batch normalization layer, a first hidden layer, a fourth batch normalization layer and a second hidden layer, wherein the first convolution layer connects to the first batch normalization layer, the first batch normalization layer connects to the second convolution layer, the second convolution layer connects to the second batch normalization layer, the second batch normalization layer connects to the first max pooling layer, the first max pooling layer connects to the first concatenate layer, the first concatenate layer connects to the second max pooling layer, the second max pooling layer connects to the first flatten layer, the first flatten layer connects to the second concatenate layer, the second concatenate layer connects to the third batch normalization layer, the third batch normalization layer connects to the first hidden layer, the first hidden layer connects to the fourth batch normalization layer, the fourth batch normalization layer connects to the second hidden layer, and wherein the second hidden layer outputs classification of individuals in the mixtures of DNA.

In another embodiment, the method further comprises a step for validating the trained deep learning model, and the trained deep learning model has accuracy equal to or more than about 90%.

In another embodiment, the method is to classify individuals in mixture of the DNAs from forensic dataset or whole exome sequencing dataset.

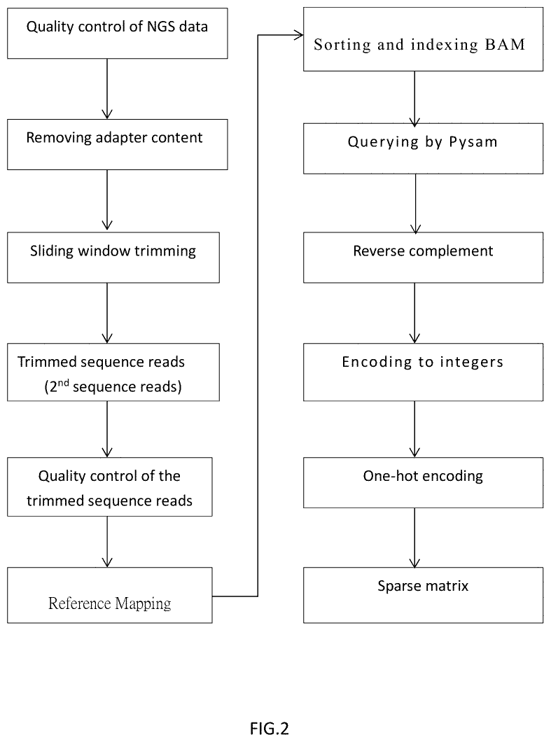

In one representative embodiment, the data processing procedure comprises following steps. The steps comprise quality control of NGS data are briefly shown in FIG. 2.

Step 1: Remove content comprises adapters from the raw sequence reads to generate first sequence reads.

Step 2: Perform a sliding window trimming on the first sequence reads to generate trimmed sequence reads with length ranging from 70 to 200 bp, and at least 25 bases are as sliding sizes for each trimming.

Step 3: Perform an examination by using phred33 score to check quality of the trimmed sequence reads; and qualified trimmed sequence reads are determined when phred33 score of the trimmed sequence reads is equal to or more than 28; or all of the trimmed sequence reads having length of 100 bp are determined to be qualified trimmed sequence reads.

Step 4: Map the qualified trimmed sequence reads onto human reference genome GRCh38 to obtain mapped sequence reads.

Step 5: Sort and index the mapped sequence reads to construct BAM files.

Step 6: Query the mapped sequence reads from the BAM files by using Pysam package.

Step 7: Perform reverse complementation to increase number of the mapped sequence reads stored in the BAM files and then generate combined forward and reverse sequence reads with length ranging from 100 to 200 bp.

Step 8: Encode the combined forward and reverse sequence reads with length ranging from 100 to 200 bp into integers by using an integer encoder; and

Step 9: Transform the integers to a plurality of sparse matrix by using one-hot encoding function, wherein the sparse matrix is constructed from the combined forward and reverse sequence reads with length ranging from 100 to 200 bp.

Moreover, the data processing comprises but not limited to following procedures.

Data Trimming

In one embodiment, to ensure high-quality reads, the raw FASTQ files are trimmed with Trimmomatic tool to remove both low-quality portions and adapter content. The raw sequence reads before and after trimming for all 20 forensic DNA mixture samples are used in the invention. For paired-end sequence data, the phred33 score is used to measure the quality of the bases. Illumina clip TruSeq2 is used for adapter content removal, and the LEADING and TRAILING functions are used to cut off bases from the start and the end, respectively, if the base quality is lower than a threshold of 3. Trimmomatic tool was used to perform trimming if the average quality of bases is below 15.

Sliding Window Trimming

In one embodiment, to display the effect of varying sequence (read) length on the performance of the DL model, if any, multiple sliding window sizes are used obtain different read lengths as inputs. To this end, sliding windows sizes of 70, 100, 150, and 200 bp are used to trim FASTQ files using the CROP and HEADCROP functions in the Trimmomatic tool. CROP and HEADCROP allow a versatile trimming function at any length from a given sequence read. For instance, to generate 4 inputs of 100 bases in length, we used 25 bases as the sliding size for each trimming time. The first sequence read is trimmed with parameters MINLEN:100 and CROP:100, resulting in sequence reads of length 100 bases. The next trimming is performed with MINLEN:125, CROP:125 and HEADCROP:25, leading to another set of sequence reads with lengths of 100 bases. The final 2 trimmings are obtained with MINLEN:150, CROP:150, and HEADCROP:50 and MINLEN:200, CROP:200, and HEADCROP:100, respectively. Parameters CROP:175 and HEADCROP:75 were bypassed to prevent excessive overlap in the middle of the reconstructed sequence reads.

Read Mapping

In one embodiment, the trimmed FASTQ files are then mapped onto the human genome reference GRCh38 using Kart version 2.5.6. Kart uses a divide-and-conquer algorithm to align reads with high speed and sensitivity. Kart identifies all locally maximal exact matches (LMEMs) from the sequence reads by searching against the reference genome using a Burrows-Wheeler transform (BWT). If the LMEMs appear multiple times in the reference sequences, they are marked as simple pair-regions (pairs with perfect alignment) which are then concatenated with normal pair-regions (un-gapped/gapped alignment) to obtain candidate alignments. Samtools version 1.3.1 is then utilized to sort and index the BAM files obtained from the mapping step, to allow rapid read querying from any genomic region. The mapped, sorted, and indexed BAM files are queried across all somatic chromosomal regions using the Alignment File and Fetch functions in Pysam and itertools. The forward sequence reads are then reverse-complemented with Bio.seq tools.

Encoding and Dimension Reduction

In one embodiment, the combined forward and reverse sequence reads obtained from the processing steps are twice encoded to transform them into a plurality of sparse matrix, a suitable input format for the neural network. Encoding the processed data in a more compact representation is a vital step. The data are mapped to a lower dimensional space by combining the data's most important features, where dimensionality reduction can decrease training time and latent feature (encoded data) representations can enhance model performance. First, the A, T, C, and G are converted into integers using an integer encoder from the Scikit-learn library, and are then transformed into a sparse matrix constructed from sequence reads of numbers using the one-hot encoding function from Scikit-learn library. The sparse matrix has 4 rows, and the column number (70-200 columns) varies according to the sequence read length (70-200 bp). These data are then ready to input into the deep learning model.

In one embodiment, after obtaining the sparse matrix for each of the combined forward and reverse sequence reads, the data were split into 80% for training, 10% for testing, and 10% for validation. The validation data was used to test the deep learning model after it had achieved the highest accuracy. Preferably, accuracy of the deep learning model is more than 90%.

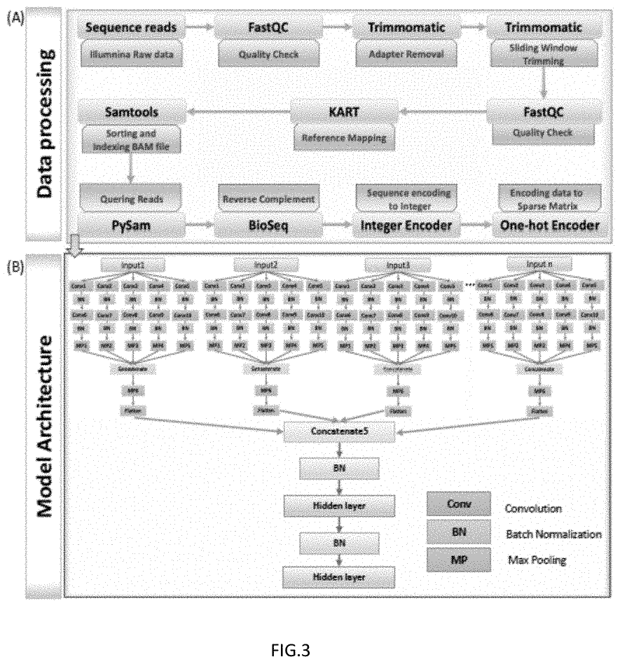

In one embodiment, workflow for the DNA ratio prediction model is shown in FIG. 3. Processing sequencing data from raw to final format is illustrated in FIG. 3(A). The raw sequencing data or reads are checked for quality control with FASTQC to detect bad reads, which are subsequently trimmed with Trimmomatic tool with different sliding window sizes ranging from 70 bp to 200 bp to generated trimmed sequence reads. These trimmed sequence reads are then mapped to the human reference genome version GRCh38 with Kart. The obtained mapped sequence reads are then sorted and indexed with Samtools. Afterward, the Pysam package is used to query the mapped sequence reads from the sorted and indexed BAM file, followed by reverse complementation with Bio.Seq to increase the number of training sequence reads. The combined forward and reverse sequence reads are encoded as integers and then undergo one-hot encoding as matrices. These matrices are used as input for the CNN. Overall schematic design of the multi-input 1-dimensional CNN is illustrated in FIG. 3B. The one-hot matrices encoding the sequence reads (i.e., sparse matrices of the combined forward and reverse sequence reads) are connected to convolutional layers with different numbers of filters ranging from 32 to 512. These convolutional layers are then connected to batch normalization layers. These two steps are repeated and followed by a max-pooling layer. In the next step, all Max-pooling data are merged into 1 concatenated layer before max-pooling again, followed by flattening. The four flattened layers are merged in a concatenation step, followed by a batch normalization layer. The data are input into the first hidden layer of the network, which includes 1024 neurons. The data from this first hidden layer are then normalized and input into the final hidden layer with a SoftMax function for classification.

DL Model Architecture

In one embodiment, the deep learning model includes 4 input blocks connected with two 1-dimensional convolution layers (CONV1D). The convolution blocks, designed with parameters consisting of learnable filters, range from 32 to 512 with a stride of 3, with padding of “same” and activation function of “relu” for 5 blocks. Stride is the moving window of the kernel for multiplication of the sequence read matrix and the kernel matrix. Padding in CNN implies adding space for kernel/filter processing, as each convolutional layer leads to reduction of the sequence space. Each layer is constructed from 5 convolutional blocks interleaved with batch normalization layers. Next, a sample-based discretization process called Max-pooling is applied. The objective of this technique is to down-sample an input representation by reducing its dimensionality while retaining the optimum information. Max-pooling (MaxPoolID) layers (5 blocks of size 3, with a stride of 2 and padding “same”) are connected to the second layer of batch normalization to extract important features from the convolutional layers. The max-pooling layers are then concatenated and undergo an additional max-pool layer followed by a flatten layer. The flatten layer transforms the matrix data into a 1-dimensional array to be used as an input into the following layers. The flattened data from 4 input blocks are then combined by another concatenate layer followed by a batch normalization layer, which is finally fed into the first hidden layer with 1024 neurons. There is one more batch normalization connected to the first hidden layer before the final hidden layer with 6 neurons is used to classify the sequence reads according to which of the 6 subjects they are from. In the case of WES data, a final hidden layer with 2 neurons is used to classify the type of breast cancer.

Training Protocols

After obtaining the sparse matrix for each of the individuals, the data were split into 80% for training, 10% for testing, and 10% for validation. The validation data was only used to test the model after it had achieved the highest accuracy.

Stochastic gradient descent was used to optimize the objective function, with a learning rate of 10e⁻⁴, a decay rate of 10e⁻⁴/10, and a momentum of 0.9 for 10 epochs for a batch size set to 128. The learning rate adjusts the adaptation/learning speed of the model. Kernel parameters with a rectified activation unit (ReLu) function were set as follows: kernel initializer “he_uniform” and kernel regularizer L2 at 0.0001. The final dense layer used a SoftMax function for final classification. Categorical cross-entropy was used to calculate the loss of model prediction and to monitor the training process by categorical accuracy metrics.

Owing to the massive number of the sequence reads in each sample, a fit generator (from Tensorflow) was used to train our models with the number of steps per epoch calculated as the total number of training samples divided by the batch size. Similarly, for the validation step, steps/epoch were calculated as the total number of validation samples divided by the batch size. After training, “predict generator” mode was used to test the model performance with the test data. The classification method reported by Scikit-learn was used to calculate and display the final classifications of each individual with weighted precision, weighted recall, and weighted F1-score. All training was done with TensorFlow version 2.0.0 (Google Inc., Mountain View, Calif.) with 2 GPU GeForce GTX 1080 Ti and 64 gigabytes of random-access memory. Measures such as accuracy, precision, recall, and F1-scores were used to evaluate the training parameters of the model (Equations 1-4).

$$Accuracy = \frac{Σ True positive + Σ True negative}{( \begin{matrix}Σ True positive + Σ True negative + \\ Σ False positive + Σ False negative\end{matrix} )} \qquad ( 1 )$$

$$Precision = \frac{Σ True positive}{Σ ( True Positive + False Positive )} \qquad ( 2 )$$

$$Recall = \frac{Σ True positive}{Σ ( ( True Positive + True Negative )} \qquad ( 3 )$$

In one embodiment, the combined forward and reverse sequence read length input into the deep learning model affects model performance because of different combined forward and reverse sequence read length. The combined forward and reverse sequence reads are transformed into sparse matrices containing coded information on all the bases in the aforementioned sequence, which would eventually generate different features. As a result, multiple sequence length combinations or combinations based on at least 4 sparse matrices generated from the combined forward and reverse sequence reads with same length or different length are preferred as inputs into the DL model for classifying individuals in the mixtures of DNA in the invention.

Effect of Sliding Window Sizes on Model Performance

In one embodiment, the invention used Trimmomatic tool to trim the raw NGS reads into multiple lengths ranging from 70 to 200 bp. An upper limit of 200 bp was chosen because beyond this length, the reads were at low quality (phred33 score<15). The results showed that for a trimmed sequence read length of 70 bp, the model accuracy was 0.36. The corresponding weighted average of precision, recall, and F1-score were 0.37, 0.36, and 0.35, respectively (Table 1). The model performance was higher for the trimmed sequence read length of 100 bp with an accuracy of 0.39 and 0.39, 0.39, and 0.38 for weighted averages of precision, recall, and F1-score, respectively. The model accuracy progressively improved with trimmed sequence read length of 150 bp and 200 bp, recording an accuracy of 0.57 and 0.67, respectively, and weighted average precision of 0.59 and 0.67, recall of 0.57 and 0.67, and F1-score of 0.57 and 0.66, respectively (Table 1). These results reaffirmed that the trimmed sequence read length plays a major role in the performance of the model and is crucial for obtaining optimal results.

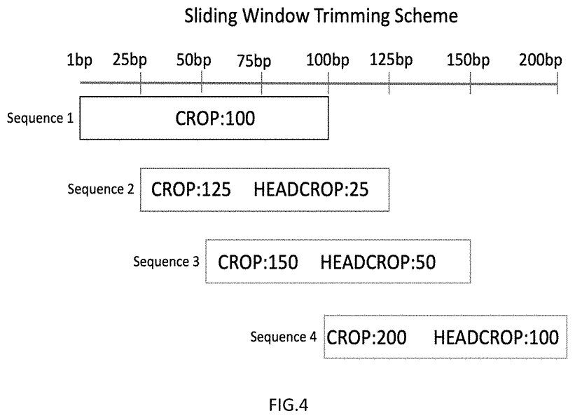

In one preferable embodiment, according to FIG. 4, to display the effect of varying sequence (read) length on the performance of the DL model, if any, multiple sliding window sizes are used to obtain different trimmed sequence read lengths as inputs. To this end, trimmed sequence read lengths of 70, 100, 150, and 200 bp are obtained from FASTQ files by using the CROP and HEADCROP functions in the Trimmomatic tool. CROP and HEADCROP allow a versatile trimming function at any length from a given sequence read. For instance, to generate 4 inputs of 100 bases in length, we used 25 bases as the sliding size for each trimming time. The first sequence read is trimmed with parameters MINLEN:100 and CROP:100, resulting in trimmed sequence reads of length 100 bases. The next trimming is performed with MINLEN:125, CROP:125 and HEADCROP:25, leading to another set of trimmed sequence reads with lengths of 100 bases. The final 2 trimmings are obtained with MINLEN:150, CROP:150, and HEADCROP:50 and MINLEN:200, CROP:200, and HEADCROP:100, respectively. Parameters CROP:175 and HEADCROP:75 were bypassed to prevent excessive overlap in the middle of the reconstructed sequence reads.

Multiple Sequence Length Combinations as Input and its Effect on Model Performance

In another preferable embodiment, the invention provides a combination of different sequence read lengths for potentially increasing an effect on the model performance. To follow this through, 3 different combinations based on 2, 3, and 4 inputs, respectively, were created as shown in Table 1. The variations for 2-input combinations included 100 bp+150 bp, 100 bp+200 bp, and 150 bp+200 bp; and 3-input combinations incorporated 70 bp+100 bp+150 bp and 100 bp+150 bp+200 bp. However, the 4-input combination was created with only 100 bp as the window size, but with different trimming strategies. The results from training the same model architecture with different combinations of inputs are list in Table 1. Intriguingly, the model performance got boosted with increase in the number of combinations of input sequence read lengths. The model performance was highest with an accuracy of 1 when the input size was set at 200 bp. For 2 inputs, the best model was obtained with a combination of sequence read lengths of 150 bp+200 bp, displaying 0.91, 0.91, 0.91, and 0.91 as the accuracy, weighted precision, recall, and F1-score, respectively. For the 3-input combination, the best model was recorded for a trimmed sequence read lengths of 100 bp+150 bp+200 bp, with 0.96, 0.96, 0.96, and 0.96 as accuracy, weighted precision, recall, and F1-score, respectively. The model that displayed the best performance (referred to as the “best model” hereafter) was for the 4-inputs combination (100 bp+100 bp+100 bp+100 bp), recording 0.97, 0.97, 0.97 and 0.97 as the accuracy, weight precision, recall, and F1-score, respectively. The precision and recall score were increased when more sequence read lengths were combined and the highest were achieved with combination of 4 sequence reads of 100 bp.

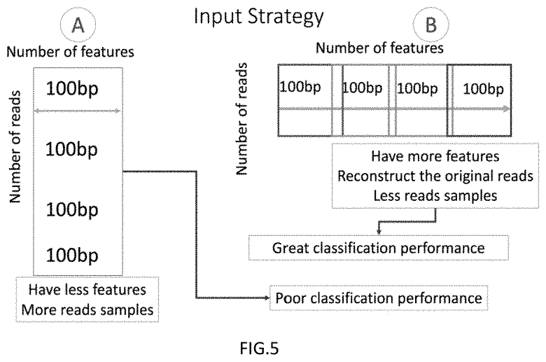

Preferably, as shown in FIG. 5, combinations based on 4 sparse matrices generated from 4 of the combined forward and reverse sequence reads with 100 bp are as inputs in the invention.

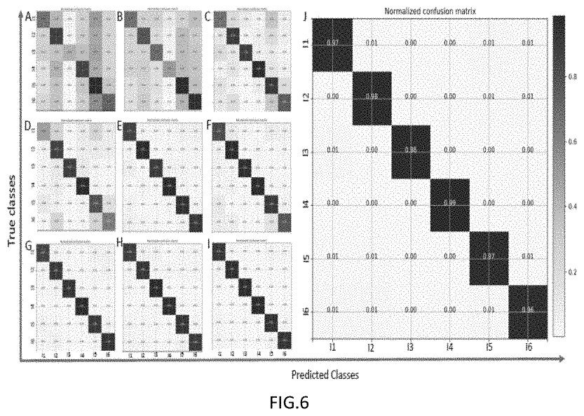

Also, the combination of different sequences read length significantly affected to model performance. The number of the sequence reads used for training the best model is displayed in Table 1. Moreover, we have also provided the confusion matrix plot of each model to observe the model performance on each class of training data as illustrated in FIG. 6 with the color of the diagonal line illustrating the closeness of the match between the predicted and the true class. The darker the blue color of the diagonal line, the better the model prediction accuracy.

The confusion matrix plot of 10 different models trained with sequence reads trimmed by different sliding windows is shown in FIG. 6. These 10 models were trained with sequence read lengths of 70 bp (A), 100 bp (B), 150 bp (C), 200 bp (D), 100 bp combined with 150 bp (E), 100 bp combined with 200 bp (F), 150 bp combined with 200 bp (G), 70 bp combined with 100 bp and 150 bp (H), 100 bp combined with 150 bp and 200 bp (I), and 4 sequence reads of 100 bp as 4 input (J). The confusion matrix displays the predicted classes on the X-axis and the true classes on the Y-axis, with the color of the diagonal line illustrating the closeness of the match between the predicted and the true class. The darker the blue color of the diagonal line, the better the model prediction accuracy.

TABLE 1
Model performance with different number of inputs

| Number of inputs/ | | Weighted | Weighted | Weighted | |
|---|---|---|---|---|---|
| sequence read length | | Ave. Accuracy | Ave. precision | Ave. recall | F1-score |
| 1 | 70 bp | 0.36/3,481,424 | 0.37 | 0.36 | 0.35 |
| | 100 bp | 0.39/1,993,376 | 0.39 | 0.39 | 0.38 |
| | 150 bp | 0.57/1,347,028 | 0.59 | 0.57 | 0.57 |
| | 200 bp | 0.67/439,288 | 0.67 | 0.66 | 0.66 |
| 2 | 100 bp + 150 bp | 0.83/1,347,028 | 0.83 | 0.83 | 0.83 |
| | 100 bp + 200 bp | 0.85/439,288 | 0.85 | 0.85 | 0.85 |
| | 150 bp + 200 bp | 0.91/439,288 | 0.91 | 0.91 | 0.91 |
| 3 | 70 bp + 100 bp + 150 bp | 0.89/2,976,580 | 0.89 | 0.89 | 0.89 |
| | 100 bp + 150 bp + 200 bp | 0.96/439,288 | 0.96 | 0.96 | 0.96 |
| 4 | 100 + 100 + 100 + 100 bp | 0.97/1,993,376 | 0.97 | 0.97 | 0.97 |

One of the important technical features described in the invention is when training a DL model is that the sample size should be consistent for all samples. In the invention, this is the sequence read length. Originally the length of all original sequence reads from the Illumina Forenseq kit (i.e., NGS reads) is 350 bp; however, the original sequence read length varied drastically once poor-quality sequence reads and adapters were trimmed off. Therefore, the sliding window trimming was performed to overcome the original sequence read length variation and maintain the consistency of sample size with a specified length. After removal of adapters and poor-quality reads, the quality score dropped extensively for read lengths≥200 bp. Therefore, the shortest and longest sliding window were set at lengths 70 bp and 200 bp (i.e., trimmed sequence reads), respectively, to retain the maximum information from the original sequence reads, simultaneously achieving high quality. Notably, the features extracted from different sliding windows also varied with the matrix size. For example, the information retained by the STRs and SNPs in the 200 bp sliding window was higher than that retained by a smaller sliding window. This discrepancy may be explained by whether sequence reads included in a sliding window contain the STR or SNP information or not. Intuitively, the longer the sequence read, the more information it retains. This point is demonstrated by the model using with 200 bp, which displayed higher accuracy relative to shorter sliding windows. This result demonstrated that read length has a crucial effect on the model performance.

Evaluation of Trained Model on Forensic Datasets

In another embodiment, evaluate the model and its ability to identify major and minor contributors in mixed DNA samples obtained from forensic datasets, we used mixed DNA samples that were manually prepared from sequence reads of 3 individuals. The mixtures were prepared with different ratios of major and minor contributors, to gauge the precision of the classification model even for very skewed mixtures. Our pre-trained model was able to identify correctly, both major and minor contributors in the mixture with either 2 major contributors or 1 major contributor. Additionally, we also prepared a mixture of DNA from 3 individuals from another replicated samples, and tested our pre-trained model performance. This replicated dataset was highly similar to the original dataset (73.4% to 91.8%) used for training. Our model was able to successfully identify all the major and minor contributors with high accuracy 80%-95%. For instance, at the mixing ratio of 1:1:1, the model predicted with high accuracy ranging from 0.90 to 0.997 for all mixture samples. Furthermore, even with highly skewed mixtures, such as ratios 9:1:1 or 9:9:1, our model could identity major and minor contributors with high confidence.

In another embodiment, for proving the deep learning model performance, an NGS data originated from 20 mixed DNA samples is prepared for the deep learning model testing. The deep learning model successfully detected major contributors in all 20 mixed samples and identified both major and minor contributors in 13 out of 20 samples. For instance, as shown in Table 2, at a mixing ratio of 1:9, the model successfully identified all (10 out of 10) of the major contributors and minor contributors in 8 out of 10 samples, and at the 1:39 mixing ratio, the model could successfully identify 100% of major contributors, but only 50% of the minor contributors.

TABLE 2

| | Predicted ratio | | | | | | | | | |
|---|---|---|---|---|---|---|---|---|---|---|
| | I1:I2 | I5:I2 | I3:I2 | I4:I2 | I3:I1 | I1:I4 | I3:I5 | I4:I5 | I5:I6 | I2:I6 |
| Real ratio | (1A-B) | (2A-B) | (3A-B) | (4A-B) | (5A-B) | (6A-B) | (7A-B) | (8A-B) | (9A-B) | (10A-B) |
| 1:9 | ** | ** | * | ** | ** | ** | ** | ** | ** | * |
| 1:39 | ** | ** | * | * | ** | ** | * | * | * | ** |

* means the model can identify major contributor
** means the model can identify both major and minor contributors

In still another embodiment, the invented method and deep learning model achieved high accuracy in identifying the major and minor contributors from an artificial mixture of 3 individuals as shown in Table 3 and 4. Mixtures from 2 major and 1 minor contributor, and from 1 major and 2 minor contributors, were created at different ratios to test the sensitivity of the trained model to a low number of sequence reads from minor contributors. The baseline numbers for minor contributors in these artificial mixtures were 34,500 and 20,000 sequence reads for the training data and the replicate samples, respectively (Table 3 and 4). The error rate of the model was noticeably very low (3%), which is equivalent to approximately 59,801 sequence reads for 6 classes (as we trained our model with −1,993,376 sequence reads). The estimated average number of incorrectly predicted sequence reads for each class was 9,966. This number includes both forward and reverse complement sequence reads. Consequently, if the number of the sequence reads from minor contributors falls below this threshold, the model might not correctly identify the minor contributors in the mixtures. Therefore, the minimum number of the sequence reads in these mixtures was set above the aforementioned threshold, which explains the high performance of the trained model when artificial mixtures are used for validation.

TABLE 3

| Mixed | I1:I2:I6(predicted | I2:I4:I1(predicted | I4:I6:I2(predicted | I5:I1:I4(predicted | I6:I4:I5(predicted | | |
|---|---|---|---|---|---|---|---|
| ratio: | ratio) | ratio) | ratio) | ratio) | ratio) | Total sequences | |
| Two major contributors | | | | | | | |
| 1:1:1 | 0.92:0.94:0.95 | 0.94:0.91:0.94 | 0.9:0.93:0.96 | 0.99:0.92:0.90 | 0.93:0.895:0.997 | 942k | (314k:314k:314k) |
| 2:2:1 | 1.83:1.9:0.98 | 1.94:1.78:0.91 | 1.78:1.82:0.83 | 1.96:1.78:0.98 | 1.86:1.79:0.85 | 786k | (314k:314k:157k) |
| 4:4:1 | 3.65:3.68:0.8 | 3.72:3.6:0.97 | 3.6:3.6:0.83 | 3.8:3.6:0.95 | 3.68:3.6:0.8 | 707.5 | (314k:314k:78.5k) |
| 8:8:1 | 7.29:7.33:0.75 | 7.44:7.12::0.82 | 7.12:7.2:0.67 | 7.66:7.15:0.975 | 7.28:7.15:0.64 | 668.25k | (314k:314k:39.25k) |
| 9:9:1 | 8:8.3:0.71 | 8.38:8.1:0.757 | 8.12:8.27:0.665 | 8.71:8.12:0.989 | 8.37:8.1:0.59 | 662.5k | (314k:314k:34.5k) |
| One major contributor | | | | | | | |
| 2:1:1 | 1.74:0.9:0.95 | 1.99:0.91:0.74 | 1.76:0.98:0.82 | 1.93:0.7:0.89 | 1.83:0.91:0.98 | 668k | (315k:157k:157k) |
| 4:1:1 | 3.47:0.9:0.945 | 3.69:0.976:0.88 | 3.52:0.98:0.89 | 1.86:0.8:0.95 | 1.83:0.93:0.92 | 511k | (314k:78.5k:78.5k) |
| 8:1:1 | 6.93:0.83:0.88 | 7.38:0.93:0.98 | 7.1:0.98:0.8 | 7.4:0.88:0.87 | 7.3:0.94:0.87 | 392.5k | (314k:39.25k:39.25k) |
| 9:1:1 | 7.8:0.81:0.86 | 8.0:0.92:0.918 | 7.9:0.97:0.79 | 8.4:0.93:0.86 | 8.15:0.94:0.77 | 384k | (314k;35k:35k) |

TABLE 4

| Mixed | I1:I2:I6(predicted | I2:I4:I1(predicted | I4:I6:I2(predicted | I5:I1:I4(predicted | I6:I4:I5(predicted | | |
|---|---|---|---|---|---|---|---|
| ratio | ratio) | ratio) | ratio) | ratio) | ratio) | Total sequences | |
| | Two major | | | | | | |
| | contributors | | | | | | |
| 1:1:1 | 0.76:0.95:0.97 | 0.95:0.88:0.76 | 0.86:0.97:0.94 | 0.99:0.75:0.87 | 0.99:0.86:0.96 | 600k | (200k:200k:200k) |
| 2:2:1 | 1.5:2:1 | 1.91:1.74:0.63 | 1.7:1.86:0.87 | 1.88:1.48:0.95 | 1.86:1.72:0.91 | 500k | (200k:200k:100k) |
| 4:4:1 | 3:3.84:0.95 | 3.87:3.38:0.69 | 1.65:1.9:0.74 | 3.8:2.97:0.996 | 3.84:3.3:0.99 | 360k | (160k:160k:40k) |
| 8:8:1 | 6:7.68:0.9 | 7.95:6.68:0.8 | 6.61:7.64:0.56 | 7.6:5.9:0.82 | 7.62:6.65:0.96 | 340k | (160k:160k:20k) |
| 9:9:1: | 6.6:8.84:0.8 | 8.66:8.99:0.87 | 7.9:8.5:0.6 | 8.4:6.62:0.79 | 8.5:7.96:0.83 | 380k | (180k:180k:20k) |
| | One major | | | | | | |
| | contributor | | | | | | |
| 2:1:1 | 1.48:0.9:0.94 | 1.96:0.94:0.62 | 1.68:0.99:0.94 | 1.84:0.6:0.94 | 1.83:0.92:0.96 | 400k | (200k:100k:100k) |
| 4:1:1 | 2.96:0.81:0.88 | 3.89:0.95:0.65 | 3.3:0.96:0.79 | 3.63:0.625:0.93 | 3.7:0.9:0.93 | 300k | (200k:50k:50k) |
| 8:1:1 | 5.93:0.57:0.94 | 7.54:0.95:0.71 | 6.6:0.92:0.67 | 7.2:0.66:1 | 7.4:0.98:0.93 | 200k | (160k:20k:20k) |
| 9:1:1 | 6.55:0.59:0.83 | 8.4:0.93:0.76 | 7.9 0.9:0.76 | 7.85:0.71:0.96 | 8.25:0.98:0.86 | 220k | (180k:20k:20k) |

In still another embodiment, the invention is related to the identification of major and minor contributors in the mixed DNA samples. The original sequence reads from minor contributors after adapter and quality control trimming ranged from 9,701 to 14,334 and 9,917 to 15,667 at the 1:9 and 1:39 mixing ratios, respectively. The number of the sequence reads from these minor contributors passed the invented model error threshold; however, the mixed DNA samples only retained 28.8% to 53.9% of their original sequence reads as shown in Table 5. Therefore, the original mixing ratio (1:9 and 1:39) as well as the sequence reads is significantly altered after the trimming process. This explain why the invented method could identify 100% of the major contributors but only 80% (1:9) and 50% (1:39) of the minor contributors in these samples.

TABLE 5
Number of sequences from raw DNA mixtures
before and after trimming

| Before | After | | Before | After |
|---|---|---|---|---|
| trimming | trimming | | trimming | trimming |
| 1A | | | 1B | |
| 346872 | 99811 | (28.8%) | 283478 | 147808 (52.14%) |
| 2A | | | 2B | |
| 190817 | 97018 | (50.8%) | 190535 | 99176 (67.21%) |
| 3A | | | 3B | |
| 369710 | 143349 | (38.8%) | 304277 | 120016 (39.44%) |
| 4A | | | 4B | |
| 289827 | 138744 | (47.8%) | 383980 | 111877 (29.13%) |
| 5A | | | 5B | |
| 241695 | 97932 | (40.5% ) | 313869 | 113765 (37.24%) |
| 6A | | | 6B | |
| 338502 | 141973 | (41.9%) | 318265 | 156678 (49.22%) |
| 7A | | | 7B | |
| 191670 | 89226 | (46.6%) | 209643 | 110599 (52.8%) |
| 8A | | | 8B | |
| 264984 | 139831 | (52.7%) | 349385 | 145971 (41.8%) |
| 9A | | | 9B | |
| 200514 | 98815 | (49.3%) | 194302 | 100428 (51.7%) |
| 10A | | | 10B | |
| 305895 | 131245 | (42.9%) | 239589 | 129154 (53.9%) |

WES Model Performance

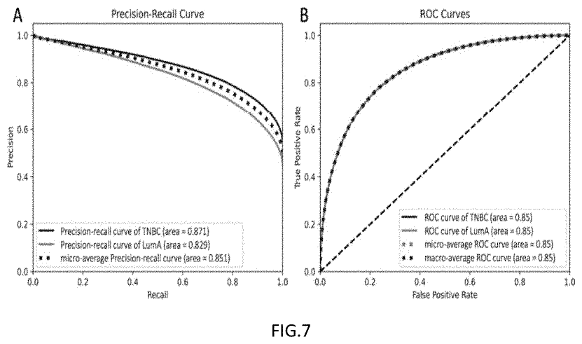

Rapid development of NGS technology allows researchers to perform large scale genomic studies at a reasonable cost. Whether selectively picking targeted genes of interest or completely profiling exome regions, these experimental approaches provide useful information for different biomedical research purposes such as classifying individuals or distinguishing different categories in case-control experiments. In still another embodiment, the invention applies deep learning (DL) model of targeted SNPs and STRs from a forensic panel. The invention further used the deep learning model to classify different subtypes of breast cancer using WES data. Targeted sequencing uses pre-defined gene information, so it is less efficient when applied to large-scale rearrangements and copy number variations. However, as WES is a comprehensive profiling method, it may lead to high similarity between individuals. In the invention, we used only chromosomes 21 and 22 from the WES data, to showcase the versatility of the model when applied to different kinds of sequencing data. Usually, mRNA expression data (PAM50, mammaprint, and blueprint) is used for breast cancer subtyping, where different gene numbers are used to classify breast cancer into luminal, basal, and HER2 subtypes (blueprint); or luminal A, luminal B, HER2, and basal subtypes (PAM50); or high and low risk subtypes. Although the genes from these methods are not all located on chromosomes 21 and 22, the invented method and DL model still distinguish luminal A from TNBC subtype, indicating that there may still be hidden genomic information and DL modeling can directly learn the differences by using sequence read information for subtype classification. Furthermore, the invention applies a WES dataset to classify patients' intrinsic molecular breast cancer subtypes. As shown in FIG. 7, the classification of TNBC and luminal A breast cancer subtype has a precision-recall curve area of 0.871 and 0.829, respectively, with a micro-average of 0.851 (FIG. 7A). In addition, we also plotted the area under the ROC curve, with an area of 0.85 for both TNBC and Luminal A (FIG. 7B). The trained model was then tested with 20 external samples, 13 TNBC and 7 luminal A. The model successfully classified 100% of the samples for each subtype (Table 6).

TABLE 6
Model predicting triple negative breast cancer and luminal A

Model prediction

| Subtypes | Sample | TNBC (%) | Luminal A (%) |
|---|---|---|---|
| TNBC | [symbol] | 76.5 | 23.5 |
| | [symbol] | 76.9 | 22.4 |
| | [symbol] | 77.6 | 23.3 |
| | [symbol] | 76.7 | 23.5 |
| | [symbol] | 76.3 | 24.0 |
| | [symbol] | 76.0 | 22.3 |
| | [symbol] | 77.3 | 23.8 |
| | [symbol] | 76.4 | 20.7 |
| | [symbol] | 79.3 | 24.8 |
| | [symbol] | 75.2 | 22.3 |
| | [symbol] | 77.7 | 22.3 |
| | [symbol] | 77.7 | 23.9 |
| | [symbol] | 77.1 | 73.9 |
| Luminal A | [symbol] | 24.7 | 75.3 |
| | [symbol] | 28.3 | 71.7 |
| | [symbol] | 26.6 | 73.4 |
| | [symbol] | 31.6 | 68.4 |
| | [symbol] | 26.3 | 73.2 |
| | [symbol] | 31.3 | 68.7 |
| | [symbol] | 26.7 | 73.3 |

[symbol]

indicates data missing or illegible when filed

In still another embodiment, the DL models trained by the sequence reads data can also be employed for other tasks such as detecting circulating tumor DNA (ctDNA) in blood samples, which is used as a biomarker for staging and prognosis, tumor localization, or drug resistance mechanisms. It is well known that cancer patients have higher amounts of cell-free DNA (cfDNA) than healthy people. The percentage of ctDNA in cancer patients, ratio of ctDNA to the normal cell free DNA (cfDNA), ranges from 0.1% to 90% depending on the type and subtype of cancer, which is highly similar to the mixed DNA samples in our forensic data. There is also great variability of the amount of ctDNA with tumor types, resulting in different detection frequency of ctDNA. Therefore, it is a great challenge to detect ctDNA within the total cfDNA in the same individual. Consequently, using sequence reads to train a model for distinguishing ctDNA from cfDNA could be a novel application of DL in the future. It would provide a minimally invasive approach for ctDNA detection. Another potential application of our pipeline is to predict the migration/metastasis ability of tumor tissue, since cancer cell migration significantly affects treatment selection. Therefore, if the model can predict the possibility of tumor cell migration in advance, it could assist physicians in making treatment decisions.

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method for classifying individuals in mixtures of DNA, comprising:
(1) Providing next-generation sequencing (NGS) data which comprises raw sequence reads originated from mixtures of DNA;
(2) Performing a data processing procedure to generate a plurality of sparse matrix; and
(3) Inputting the plurality of sparse matrix into a trained deep learning model installed on computers to classify individuals in the mixtures of DNA.

2. The method according to claim 1, wherein the data processing procedure comprises following steps:
(1) removing a content comprises adapters from the raw sequence reads to generate first sequence reads;
(2) Performing a sliding window trimming on the first sequence reads to generate trimmed sequence reads with length ranging from 70 to 200 bp, and at least 25 bases are as sliding sizes for each trimming;
(3) Performing an examination by using phred33 score to check quality of the trimmed sequence reads; and qualified trimmed sequence reads are determined when phred33 score of the trimmed sequence reads is equal to or more than 28; or all of the trimmed sequence reads having length of 100 bp are determined to be qualified trimmed sequence reads;
(4) Mapping the qualified trimmed sequence reads onto human reference genome GRCh38 to obtain mapped sequence reads;
(5) Sorting and indexing the mapped sequence reads to construct BAM files;
(6) Querying the mapped sequence reads from the BAM files by using Pysam package;
(7) Performing reverse complementation to increase number of the mapped sequence reads stored in the BAM files and then generate combined forward and reverse sequence reads with length ranging from 100 to 200 bp;
(8) Encoding the combined forward and reverse sequence reads with length ranging from 100 to 200 bp into integers by using an integer encoder; and
(9) Transforming the integers to a plurality of sparse matrix by using one-hot encoding function, wherein the sparse matrix is constructed from the combined forward and reverse sequence reads with length ranging from 100 to 200 bp.

3. The method of claim 1, further comprises a step for checking quality of the raw sequence reads, and phred33 score is used for measure of the quality of the raw sequence reads, and the raw sequence reads are trimmed if the phred33 score is below 15.

4. The method of claim 1, wherein the trained deep learning model is a one-dimensional deep convolutional neural network constructed from a first convolution layer, a first batch normalization layer, a second convolution layer, a second batch normalization layer, a first max pooling layer, a first concatenate layer, a second max pooling layer, a first flatten layer, a second concatenate layer, a third batch normalization layer, a first hidden layer, a fourth batch normalization layer and a second hidden layer, wherein the first convolution layer connects to the first batch normalization layer, the first batch normalization layer connects to the second convolution layer, the second convolution layer connects to the second batch normalization layer, the second batch normalization layer connects to the first max pooling layer, the first max pooling layer connects to the first concatenate layer, the first concatenate layer connects to the second max pooling layer, the second max pooling layer connects to the first flatten layer, the first flatten layer connects to the second concatenate layer, the second concatenate layer connects to the third batch normalization layer, the third batch normalization layer connects to the first hidden layer, the first hidden layer connects to the fourth batch normalization layer, the fourth batch normalization layer connects to the second hidden layer, and wherein the second hidden layer outputs classification of individuals in the mixtures of DNA.

5. The method of claim 1, further comprises a step for validating the trained deep learning model, and the trained deep learning model has accuracy equal to or more than about 90%.

6. The method of claim 1, being to classify individuals in mixture of the DNAs from forensic dataset or whole exome sequencing dataset.
